(19) United States
(12) Patent Application Publication
(10) Pub. No.: US 2019/0142571 A1
(43) Pub. Date: May 16, 2019

(54) BRANCHING COVERED STENT-GRAFTS AND RELATED DEPLOYMENT SYSTEMS AND METHODS

(71) Applicants: Derrick Chu, Maple Grove, MN (US); David C. Swanson, Saint Paul, MN (US)

(72) Inventors: Derrick Chu, Maple Grove, MN (US); David C. Swanson, Saint Paul, MN (US)

(21) Appl. No.: 16/185,195

(22) Filed: Nov. 9, 2018

Related U.S. Application Data
(60) Provisional application No. 62/585,269, filed on Nov. 13, 2017.

(51) Int. Cl. A61F 2/07; A61L 31/04; A61L 31/06; A61M 25/00; A61M 25/09

(52) U.S. Cl. CPC A61F 2/07; A61L 31/048; A61L 31/06; A61F 2002/065; A61M 25/0041; A61M 25/09; A61F 2002/072; A61M 25/0071

(57) ABSTRACT

A bifurcated stent-graft and stent-graft delivery system for placement and delivery into an anatomical structure of the body, such as the superior vena cava, are disclosed. The stent-graft includes a first, second and third wire portions that provide strength, flexibility, and resilience. The wire portions are incorporated and joined by a graft fabric that forms a flexible joint that provides increased flexibility. The stent-graft can also include an aperture to allow for unobstructed blood flow from branched or tributary blood vessels such as the azygos vein.

### RELATED APPLICATION

The present application claims the benefit of U.S. Provisional Application No. 62/585,269 filed Nov. 13, 2017, which is hereby incorporated herein in its entirety by reference.

### TECHNICAL FIELD

Embodiments relate generally to medical devices and methods, and more particularly to structures and deployment of bifurcated covered stent-grafts to improve or restore blood flow in the superior vena cava (SVC) with an aperture to preserve flow through the azygos vein.

### BACKGROUND

Stenotic or occlusive lesions can be caused by a wide variety of disease processes, including benign conditions such as those caused by prolonged indwelling central venous catheters in dialysis patients and malignancies where the blood vessel is narrowed or occluded either by direct cancer invasion or extrinsic compression by tumor mass. Depending upon the location of the stenotic or occlusive lesion, a variety of medical conditions can occur. For example, a lesion in the superior vena cava (SVC) can reduce or prevent the venous blood return from both arms and head and neck from returning to the right atrium and cause undesired symptoms including congestion and edema of the face and thorax, difficulty breathing, severe headaches, and cerebral venous hypertension. Therefore, maintaining or restoring the patency of blood vessels is of interest in the treatment of such lesions. The placement of stents and stent-grafts to maintain the patency of blood vessels is a commonly practiced, minimally invasive surgical procedure for the treatment of stenotic lesions. Generally, stenting is an effective palliative treatment for patients with a stenotic lesion obstructing the flow of venous blood or maintaining vessel patency after an occlusive lesion has been opened. Various stents and stent-grafts, which can be delivered by a variety of delivery techniques, have been developed to address these concerns.

Conventional covered stent-grafts provide a prosthetic intraluminal wall designed to oppose the patient's inner vascular walls and provide an unobstructed conduit for blood flow within the stent-graft lumen. Typically, conventional covered stent-grafts have a hollow metallic structure forming a straight, cylindrical tube coated with a biocompatible material and have terminal ends that are generally perpendicular to the cylinder's longitudinal axis. Straight, cylindrical stent-grafts have been particularly useful and effective for treatment of stenosis of vessels affected by disease. The use of straight, cylindrical stent-grafts, however, has some limitations in treatments occurring at or near a branch of a bifurcation or branching point of a blood vessel. Treatment of a bifurcation lesion has lower procedural success rates and suboptimal clinical outcomes than treatment of a non-bifurcation lesion.

One method for treating a bifurcation lesion is Y-stenting. Y-stenting techniques use two straight cylindrical stent-grafts arranged in a Y-shape with a main stent-graft to bridge a bifurcated blood vessel. One of the two stent-grafts is generally shorter and forms one leg that is attached to a longer stent-graft that forms the other leg and the common trunk of the Y-shape. A major problem with conventional Y-stenting and other techniques using straight cylindrical stent-grafts is that the site of the junction of the separate stent-graft segments can disrupt blood flow and induce thrombus formation.

Bifurcated woven metal stent-grafts have been developed to address some of the problems caused by using straight cylindrical stent-grafts at a bifurcation of a blood vessel. Generally, conventional bifurcated woven metal stent-grafts are made from a woven wire mesh forming a common trunk and two branches. Conventional bifurcated stent-grafts decrease the risk of thrombosis that is associated with the use of straight cylindrical stent-grafts at a bifurcation of a blood vessel. These conventional woven metal bifurcated stent-grafts are not wholly satisfactory for use at some bifurcations, such as at the SVC, because bifurcated stent-grafts lack the design flexibility required for adapting to a wide range of patient anatomies. Existing bifurcated stent-grafts can also obstruct blood flow from branched or tributary blood vessels that are connected at or near the bifurcated blood vessel.

Furthermore, the delivery of bifurcated stent-grafts to the treatment site is complicated by the fact that each of the trunk and two legs of the stent-graft must be positioned into their respective lumen of the bifurcated blood vessel. The bifurcated stent-grafts generally must be compressed into a very small diameter or transverse profile to facilitate intraluminal delivery through a body's tortuous vasculature. Many existing bifurcated stent-graft delivery systems have relatively large profiles, greater than desired stiffness, and are difficult to accurately deploy at the desired location.

Thus, there remains a need for improved bifurcated stent-grafts and bifurcated stent-graft delivery systems to address the problems that arise when treating a stenotic lesion at a bifurcated blood vessel.

### SUMMARY

According to an embodiment, a branched covered stent-graft suitable for implantation into an anatomical structure includes a first wire portion defining a first flowpath, a second wire portion defining a second flowpath, a third wire portion defining a third flowpath, and a flexible joint. The flexible joint is comprised of a fluid barrier material. The fluid barrier material can include Polytetrafluoroethylene (PTFE) or Polyethylene terephthalate (PET). The flexible joint defines a plenum that is fluidically couple to each of the first flowpath, second flowpath, and the third flowpath. The fluid barrier material is bonded, affixed, or otherwise coupled to the first, second, and third wire portions to form a first branch, second branch, and trunk of the branched covered stent-graft.

In embodiments, the fluid barrier material of the third wire portion, as well as the third wire portion itself in some embodiments, defines an aperture. In embodiments, the edge of the aperture begins between 0.1 centimeters (cm) and 7 cm from a junction of the flexible joint. In other embodiments, the aperture can be similarly located and defined in the second wire portion or the first wire portion. In still other embodiments, more than one aperture can be formed in the stent-graft. The aperture can be in fluid communication with the stent-graft flowpaths.

In embodiments, the fluid barrier material of the third wire portion and the third wire portion has an aligned aperture formed by the same edge. In embodiments, the edge of the aperture begins between 0.1 centimeters (cm) and 7 cm from a junction of the flexible joint. In other embodiments, the aperture can be similarly located and defined in the second wire portion or the first wire portion. In still other embodiments, more than one aperture can be formed in the stent-graft. The aperture can be in fluid communication with the stent-graft flowpaths.

In embodiments, a stent-graft delivery system is capable of deploying a branched covered stent-graft in an anatomical structure of the body. The stent-graft delivery system can deploy a stent-graft having a first wire portion defining a first flowpath, a second wire portion defining a second flowpath, a third wire portion defining a third flowpath, and a flexible joint. In embodiments, the stent-graft can include an aperture. In embodiments, the stent-graft delivery system comprises a catheter having a central shaft, a split distal portion that forms a first carrier with a lumen and a second carrier with a lumen, the first carrier and second carrier operably coupled to the stent-graft. The lumens of the first and second carriers move over a first guide wire and a second guide wire. The stent-graft is compressed by a plurality of suture loops arranged along the stent-graft. The suture loops are secured about the stent-graft by one or more pull members. The one or more pull members are operably coupled to a control mechanism. Removal of the pull members releases the suture loops from about the stent-graft, causing the stent-graft to expand from a compressed state into an expanded state. In embodiments, the tips of the first and second carrier component form a split graduated tapered nose cone. The graduated nose cone allows introducing the device through vasculature and delivering to intended target vessel with ease.

In embodiments, the stent-graft delivery system deploys the stent-graft in an anatomical structure of the body. In embodiments, the stent-graft delivery system can deploy the stent-graft at or near the bifurcation of the SVC. The first branch or second branch can be positioned at or near the left or right brachiocephalic veins. The trunk of the branched covered stent-graft can be positioned at the SVC. The flexible joint can be positioned at the bifurcation of the SVC. The bifurcated stent-graft can simultaneously support both vascular walls of the brachiocephalic veins and the vascular walls of the SVC. The stent-graft with a flexible joint comprised of a fluid barrier material or graft fabric can have increased flexibility as compared to a stent-graft with a joint comprised of a wire portion. This increased flexibility of the joint can allow each of the individual segments of the joint to be independently adjustable to fit varied geometries of the blood vessels of patients. The flexible wire portions can independently expand to fit varied geometries of the blood vessels of patients.

In embodiments, the aperture can be positioned at or near the azygos vein. The azygos vein, which transports blood to the SVC, drains itself into the SVC at or near the bifurcation of the SVC. In embodiments, the aperture is oval in shape oriented longitudinally along the SVC to accommodate variations of azygos vein position in relation to the SVC bifurcation. In embodiments, the aperture is located posterior to the plane formed by the first wired portion and the second wired portion, approximating the normal anatomic branching pattern of the SVC. In embodiments, the aperture placed at or near the azygos vein can self-align with the azygos vein to allow blood flow from the azygos vein to enter through the aperture into the stent-graft. The bifurcated covered stent-graft with an aperture placed at or near the azygos vein for the treatment of a stenotic or occlusive lesion can preserve blood flow from the azygos vein into the SVC and eliminate the need to sacrifice one of the largest collateral pathway for blood return as compared to a covered stent-graft without an aperture placed at or near the azygos vein.

In embodiments, the stent-graft and the stent-graft delivery system are operably packaged together in a kit. The kit may also include a vascular sheath to introduce the stent-graft graft and stent-graft delivery system into the body. Packaging the stent-graft and stent-graft delivery system together in a kit facilitates the use of the device by a medical professional for treatment of a patient. The kit can further include instructions for use, such as instructions for operating the stent-graft delivery system to deliver the stent-graft. Such instructions can facilitate the use of a stent-graft and stent-graft delivery systems to deploy a stent-graft in an anatomical structure of the body.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1A is a perspective view of a covered stent-graft according to an embodiment.

FIG. 1B is a partially cutaway view of the stent-graft of FIG. 1A.

FIG. 2A is a cutaway view of the stent-graft of FIG. 1A depicting the wired portions thereof.

FIG. 2B is a chart depicting types of wire mesh according to embodiments.

FIG. 3 is a perspective view of a covered stent-graft according to an embodiment.

FIG. 4 is a perspective view of a stent-graft with an aperture in the fluid barrier according to an embodiment.

FIG. 5 is a partially cutaway perspective view of a covered stent-graft with an aperture in both the fluid barrier and the wire portion according to an embodiment.

FIG. 6 is a partially cutaway perspective view of a covered stent-graft with an aperture in the fluid barrier according to an embodiment.

FIG. 7 is a partially cutaway perspective view of a covered stent-graft with an aperture in both the fluid barrier and the wire portion according to an embodiment.

FIGS. 8A and 8B are perspectives view of a stent-graft and a stent-graft delivery system, respectively, according to an embodiment.

FIGS. 8C and 8D are partial views of the stent-graft delivery system of FIG. 8A.

FIGS. 8E-8I are perspective views of the stent-graft and stent-graft delivery system of FIGS. 8A and 8B being delivered and deployed in a blood vessel.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

Embodiments described herein relate to flexible bifurcated covered stent-grafts to facilitate blood flow in the SVC or other bifurcated or branched blood vessels and stent-graft delivery systems to facilitate accurate placement of the stent-graft at the treatment site.

In order to treat a wide range of patient anatomies, it is advantageous to have a branched covered stent-graft with increased design flexibility that can be safely and accurately deployed with a stent-graft delivery system. A lack of flexibility can lead to improper alignment with the natural anatomy of the vasculature of a patient. In particular, at least some stent-graft flexibility is necessary when the stent-graft is being placed at the bifurcation of the SVC due to the unique structure of the blood vessels.

Additionally, embodiments described herein do not obstruct blood flow from branched or tributary blood vessels. For example, a stent-graft used in the SVC does not obstruct the azygos vein blood flow therefore preserving an important collateral pathway, the importance of which can be paramount in the event of treatment failure.

Furthermore, inaccurate placement of a stent-graft can reduce the effectiveness of the treatment and result in thrombosis and other negative effects. Improvements to bifurcated stent-grafts and corresponding delivery systems facilitate accurate placement, improving blood flow and increasing the efficacy of the treatment.

A branching stent-graft 100 generally comprises a first wire portion 102, a second wire portion 104, a third wire portion 106, and a flexible joint 108. Each of the three wire portions (102, 104, and 106), and/or the flexible joint 108, can comprise a fluid barrier material. First wire portion 102, second wire portion 104, and third wire portion 106 are each generally cylindrical in shape. First wire portion 102 defines proximal end 112 and distal end 114. Second wire portion 104 defines proximal end 116 and distal end 118. Third wire portion 106 defines proximal end 120 and distal end 122. Wire portions 102, 104, 106 each have a diameter in the range of 5 mm (0.20 inch) to 32 mm (1.26 inches), or in some embodiments between about 15 mm (0.59 inches) to 24 mm (0.94 inches). In embodiments, third wire portion 106 has a larger diameter than first wire portion 102 and second wire portion 104. In other embodiments, the diameter of each wire portion can be varied to fit the anatomical structure of individual patients. Wire portions 102, 104, 106 each have a length in the range of 10 mm (0.39 inch) to 70 mm (2.76 inches), the length measured being the distance between a wire portion's proximal end and distal end. Flexible joint 108 is the portion of the fluid barrier that does not have underlying wired portions, and that is interposed between each of first wire portion 102, second wire portion 104, and third wire portion 106.

In embodiments, each of the wire portions can be formed as either a single continuous wire portion, or as a superstructure of multiple wires or other components. Wire portions 102, 104, 106 can comprise one or more of nitinol, stainless steel, FePt, FePd, FeNiCoTi, FeNiC, FeMnSi, FeMnSiCrNi, or other metal or shape memory alloy materials, in embodiments. These metal or shape memory alloy materials can provide increased strength when compared to using polymers.

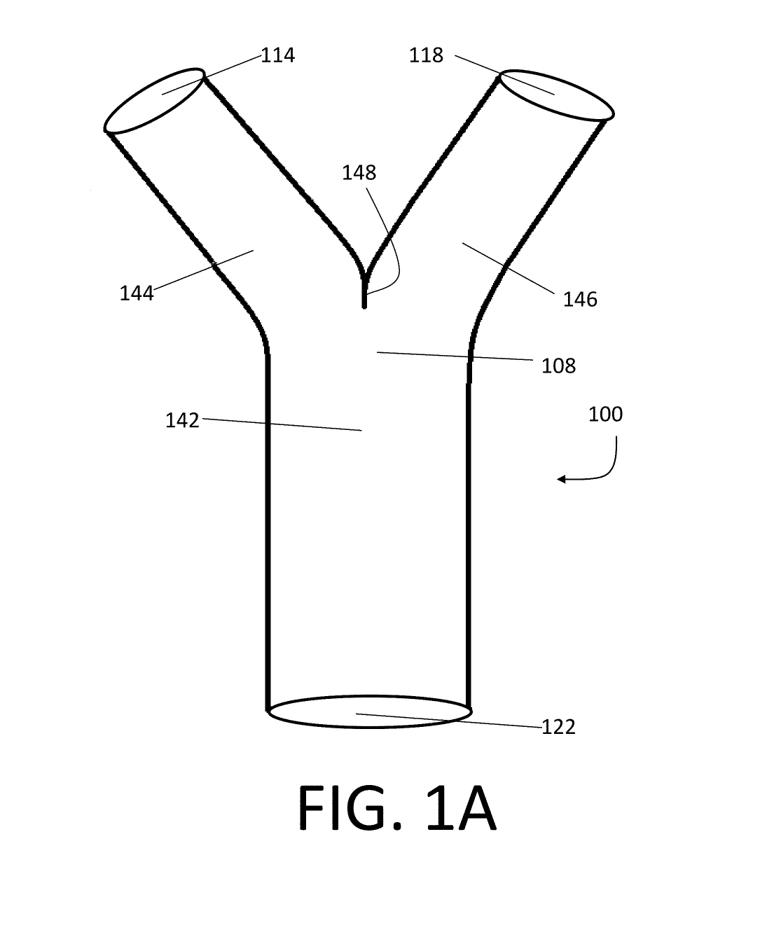

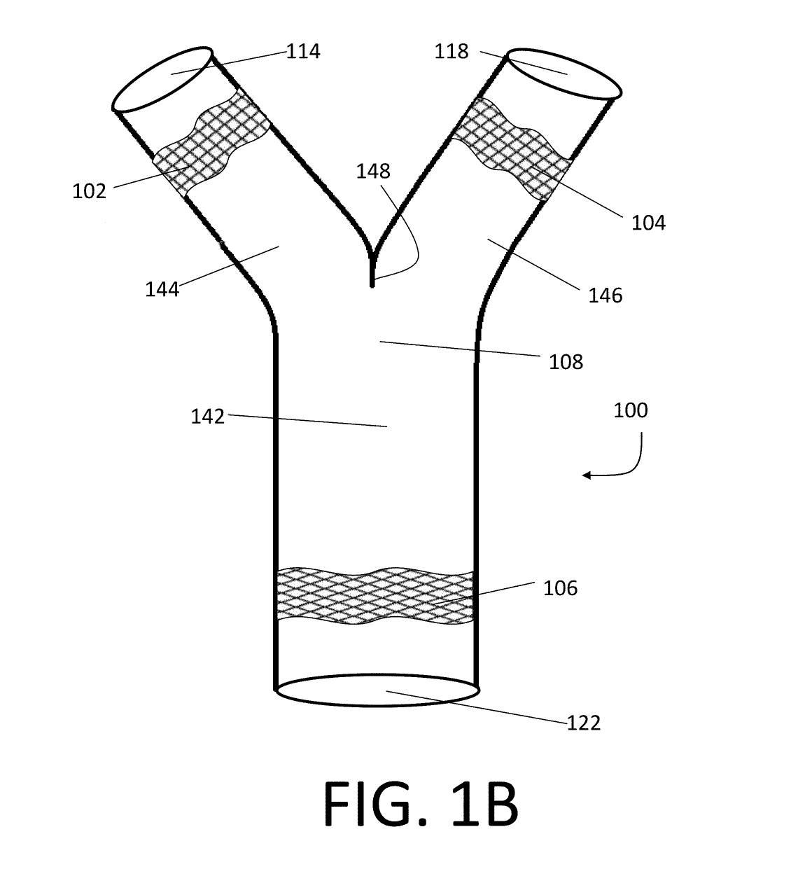

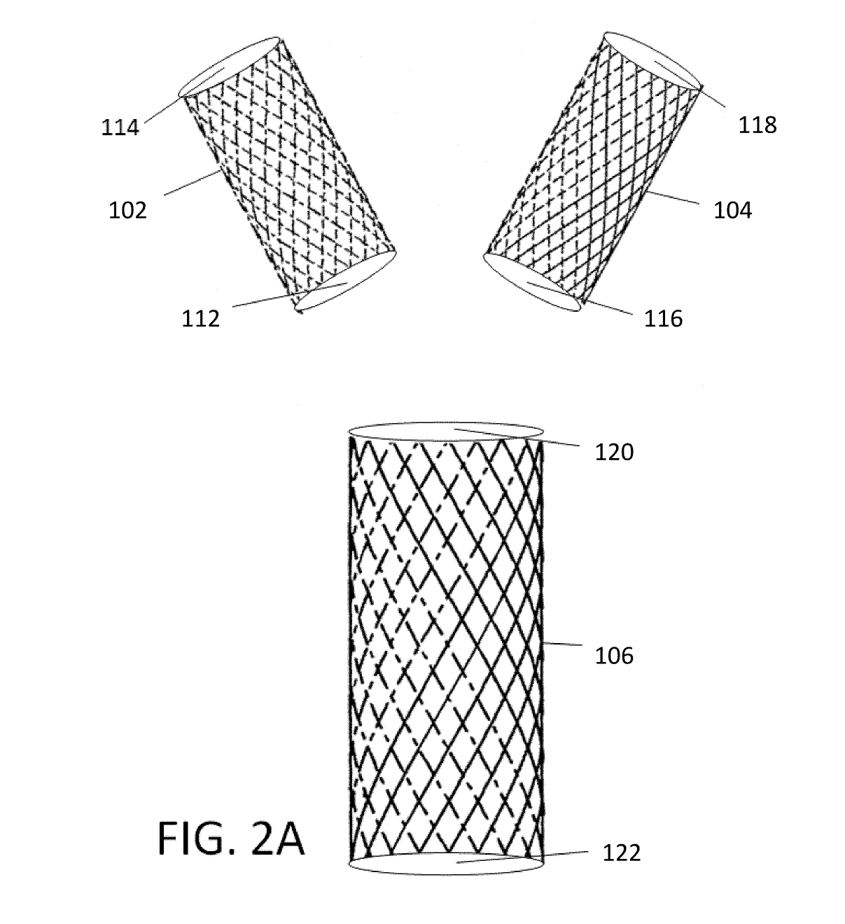

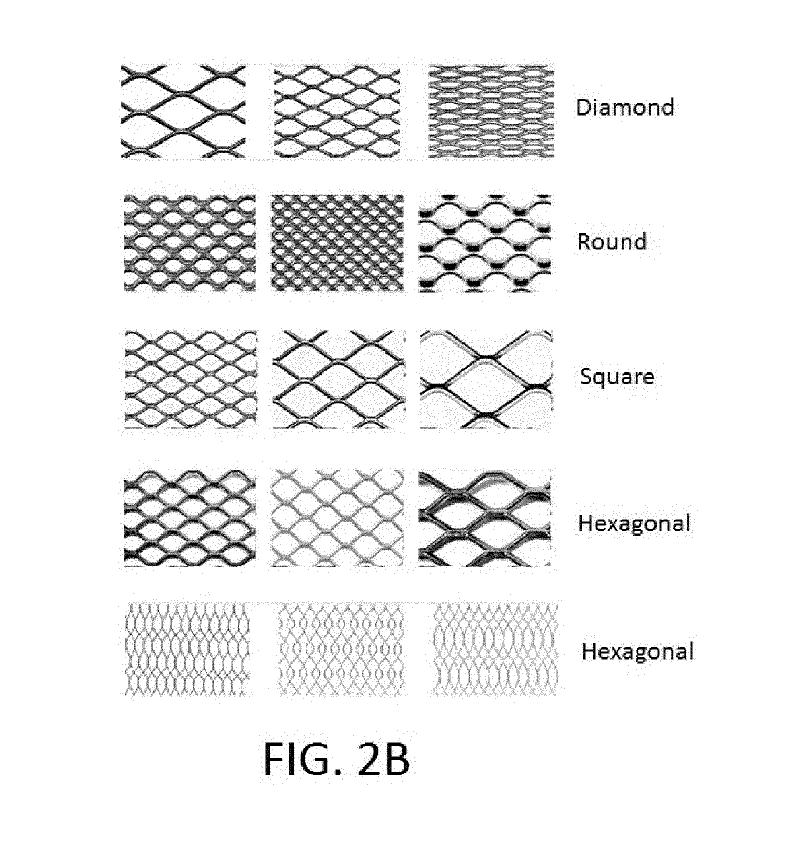

Referring to FIGS. 1A-2A, first wire portion 102 defines a flowpath from distal end 114 towards proximal end 112. Second wire portion 104 defines a flowpath from distal end 118 towards proximal end 116. Third wire portion 106 defines a flowpath from proximal end 120 towards distal end 122. Flexible joint 108 defines a plenum that is fluidically coupled to each flowpath. In embodiments, flowpaths can allow blood to flow from the distal ends 114, 118 through stent-graft 100 to distal end 122 and towards the heart when used at the SVC.

In alternative embodiments where stent-graft 100 is not used at an SVC, blood flow could be in the opposite direction. For example, the aorta distributes oxygenated blood through a system of branching arteries and vessels, and stent-grafts placed in the aortic system can be configured for blood that flows in the opposite direction, from distal end 122, through branched covered stent-graft 100 to distal ends 114, 118. In embodiments, stent-graft 100 can include one or more valves arranged to allow blood flow in a desired direction only.

In FIGS. 1A-2B, the dimensions, arrangements, sizes, and ratios shown in the drawings can be varied. FIGS. 1A-2B are not necessarily drawn to scale. In other embodiments than those specifically depicted in the drawings, the relative sizes and arrangements of the described components can be modified while still providing a suitable fluid barrier material that routes blood flow in a desired fashion. Likewise, the remainder of the drawings described in detail below may not necessarily be to scale, and the shapes and sizes of the components may not be exactly as depicted in other embodiments.

Referring to FIG. 2B, in embodiments, each of the wire portions 102, 104, 106 can be formed from wire mesh, the wire mesh including but not limited to diamond-shaped, round-shaped, square-shaped, hexagonal-shaped, or some other suitably shaped wire mesh. Referring again to FIGS. 1A-2A, cylindrical wire portions 102, 104, 106 are flexible and can be bent or flexed both radially and axially in response to a force thereon. Wire portions 102, 104, 106 can axially bend or angle to facilitate navigation through the vasculature of the body as well as facilitate deployment at an anatomical structure of the body. The bend angle created when bending wire portions 102, 104, 106 is an acute angle. Cylindrical wire portions 102, 104, 106 are configured to radially compress in a direction perpendicular to the cylinder's longitudinal axis in response to a force thereon. The diameters of wire portions 102, 104, 106 decrease when compressed. Cylindrical wire portions 102, 104, 106 are compressed in a direction perpendicular to the cylinder's longitudinal axis to facilitate the deployment of the stent-graft by a delivery system. Wire portions 102, 104, 106 possess sufficient innate resiliency and restorative power to return to an expanded position after the force thereon is released. The flexibility and resiliency of wire portions 102, 104, 106 can facilitate deployment of the stent-graft in an anatomical structure of the body as well as enhance the stent-graft's ability to maintain its conformability and its position when deployed. The wire portions 102, 104, 106 also provide strength to maintain the patency of the branched covered stent-graft.

In embodiments, flexible joint 108 is made of a fluid barrier material and is interposed between trunk 142, first branch 144, and second branch 146. First branch 144 and second branch 146 merge at junction 148. Trunk 142 and branches 144, 146 incorporate the three wired portions 102, 104, and 106. In embodiments, first branch 144 comprises first wire portion 102 and fluid barrier material that is bonded, affixed, or otherwise coupled to the exterior surface of first wire portion 102. In embodiments, second branch 146 comprises second wire structure 104 and fluid barrier material that is bonded, affixed, or otherwise coupled to the exterior surface of second wire portion 104. In embodiments, trunk 142 comprises third wire portion 106 and fluid barrier material that is bonded, affixed, or otherwise coupled to the exterior surface of third wire portion 106. Flexible joint 108 is the portion of fluid barrier that does not have an adjacent wired portion, and is interposed between each of first wire portion 102, second wire portion 104, and third wire portion 106. Flexible joint 108 comprises the same fluid barrier material that comprises trunk 142 and branches 144, 146. In embodiments, the fluid barrier material could be plastic, extruded, or contoured material. In other embodiments, the fluid barrier material can comprise PTFE or PET, for example, or any other polymeric material, non-woven material, graft fabric, or liquid barrier.

The incorporation of each of the wire portions 102, 104, and 106 into the fluid barrier fabric can be made in various ways. For example, in embodiments each of the wire portions 102, 104, and 106 is sleeved within or chemically, mechanically or thermally bonded, affixed, or otherwise coupled to the fluid barrier material. In still further embodiments, a wire portion can be operably coupled to a graft fabric by a stitching, coating, overmolding, heat application or other process.

Flexible joint 108 does not include a wire portion or scaffold. Therefore, flexible joint 108 forms a gap or discontinuity in the wire superstructure formed by wire portions 102, 104, and 106. Flexible joint 108 can have a flexible and resilient nature that allows flexible joint 108 to flex, bend, and/or fold. Flexible joint 108 can have increased flexibility and resiliency as compared to a joint with a wire portion or scaffold. The flexibility and resiliency of flexible joint 108 can facilitate deployment of the stent-graft in an anatomical structure of the body as well as enhance the stent-graft's ability to maintain its conformability and its position when deployed.

In embodiments, the angle created at junction 148 between first branch 144 and second branch 146 can be designed to fit at the bifurcation of the SVC created by the left and right brachiocephalic veins. A physician or other healthcare provider could customize a branching stent 100 to a particular vasculature, in embodiments. In embodiments, the angle created at junction 148 between first branch 144 and second branch 146 can be designed to fit other bifurcated or branched blood vessels.

While the depicted embodiment is configured for use in the SVC, similar embodiments can be designed for other bifurcated blood vessels. Depending upon the specific size and shape of the bifurcated blood vessel, the radii of each of the first wire portion 102, second wire portion 104, and third wire portion 106 can be modified, as can the angles at which they intersect at flexible joint 108.

In some embodiments, first wire portion 102, second wire portion 104, third wire portion 106, and flexible joint 108 can have different structural, material and/or other properties from each other. The different properties can include, for example, flexibility and resiliency, which can be achieved by constructing the components from different material. The embodiments depicted in FIGS. 1A-2B can enhance the stent-graft's ability to maintain its conformability and its position when deployed as compared to conventional bifurcated covered stent-grafts due to the increased flexibility and resiliency of wire portions 102, 104, 106 and flexible joint 108. Furthermore, the depicted arrangement can better fit varied geometries of the blood vessels of patients as compared to existing bifurcated stent-grafts.

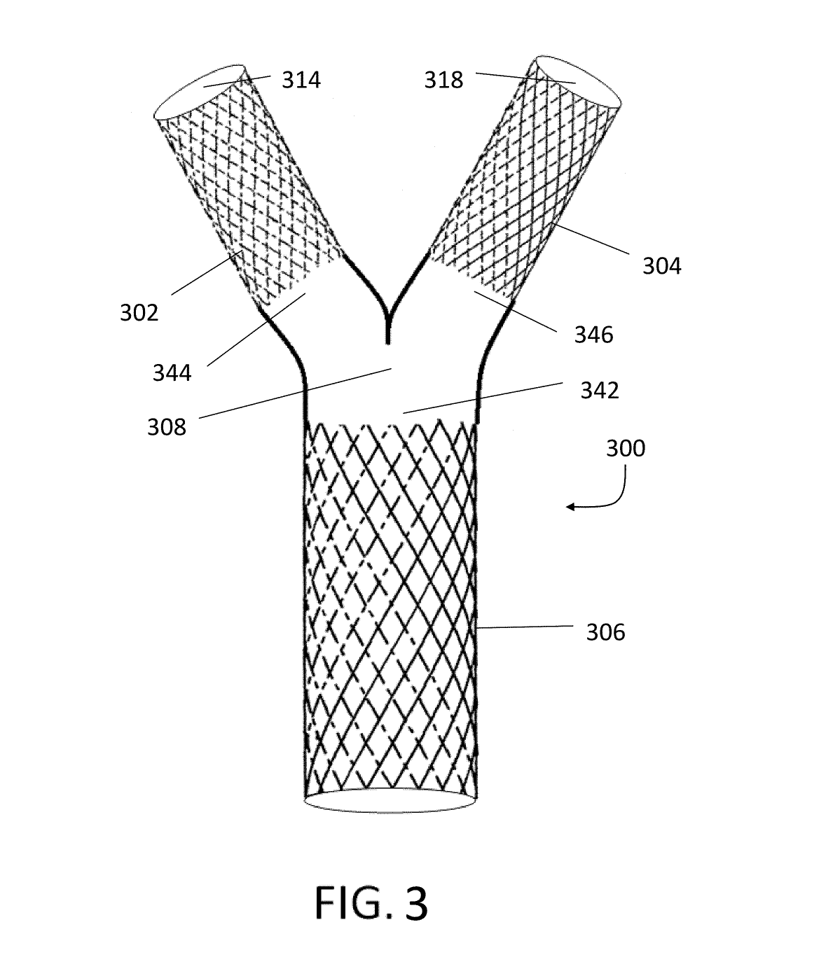

FIGS. 3-8I depict various embodiments of a branching covered stent-graft and branching covered stent-graft delivery system. Like elements are arranged similarly to the depiction of branching covered stent-graft 100 described above with respect to FIGS. 1A-2B, with reference numerals corresponding to like parts iterated by factors of 100.

FIG. 3 depicts an embodiment of a branching covered stent-graft 300. Branching covered stent-graft 300 comprises first wire portion 302, second wire portion 304, third wire portion 306, flexible joint 308, trunk 342, first branch 344, and second branch 346. In embodiments, first branch 344 comprises first wire portion 302 and a fluid barrier material that is bonded, affixed, or otherwise coupled to the interior surface of first wire portion 302. In other embodiments, described in more detail below, the fluid barrier material can be coupled to the exterior surface of the wire portions, or the wire portions can be embedded within the fluid barrier material. In embodiments, a fluid barrier material may be secured to a wire portion by mechanical, chemical or thermal bonding.

In embodiments, second branch 346 comprises second wire portion 304 and a fluid barrier material that is bonded, affixed, or otherwise coupled to the interior surface of second wire portion 304. In embodiments, trunk 342 comprises third wire portion 306 and a fluid barrier material that is bonded, affixed, or otherwise coupled to the interior surface of third wire portion 306. Flexible joint 308 is the portion of fluid barrier that does not have an adjacent wired portion, and is interposed between each of first wire portion 302, second wire portion 304, and third wire portion 306.

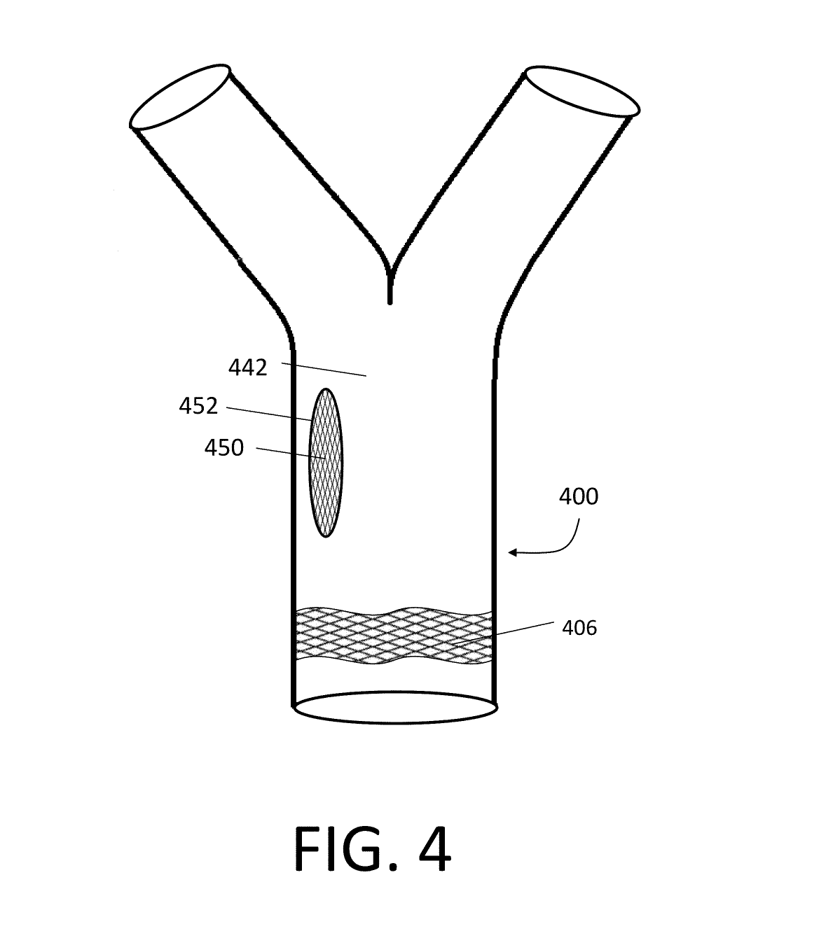

FIG. 4 depicts an embodiment of a branching covered stent-graft 400 with an aperture and a fluid barrier material bonded, affixed, or otherwise coupled to an exterior surface of wired portion 406, such as the embodiment depicted in FIGS. 1A-2A. In embodiments, a fluid barrier material may be secured to a wire portion by mechanical, chemical or thermal bonding.

In embodiments, branching covered stent-graft 400 includes third wire portion 406 and trunk 442 with an aperture 450 in the fluid barrier material of trunk 442. FIG. 4 shows a partial cut away view of the fluid barrier material of trunk 442 to show underlying third wire portion 406. An edge 452 defines aperture 450. Aperture 450 is defined within the fluid barrier material to permit blood flow from a tributary blood vessel into or out of branching covered stent-graft 400, such as to accept blood flow from the azygos vein.

In embodiments, the shape of aperture 450 is an elongated oval oriented parallel to the long axis of the cylindrical stent-graft, which allows limited variety of premade branching covered stent-graft 400 to accommodate majority of patient anatomies. In embodiments, edge 452 can include reinforced support to prevent tearing or movement of the graft fabric, such as a grommet or thickening of the underlying wire superstructure. The reinforced support of edge 452 can also provide a smoothed edge that can reduce tissue damage.

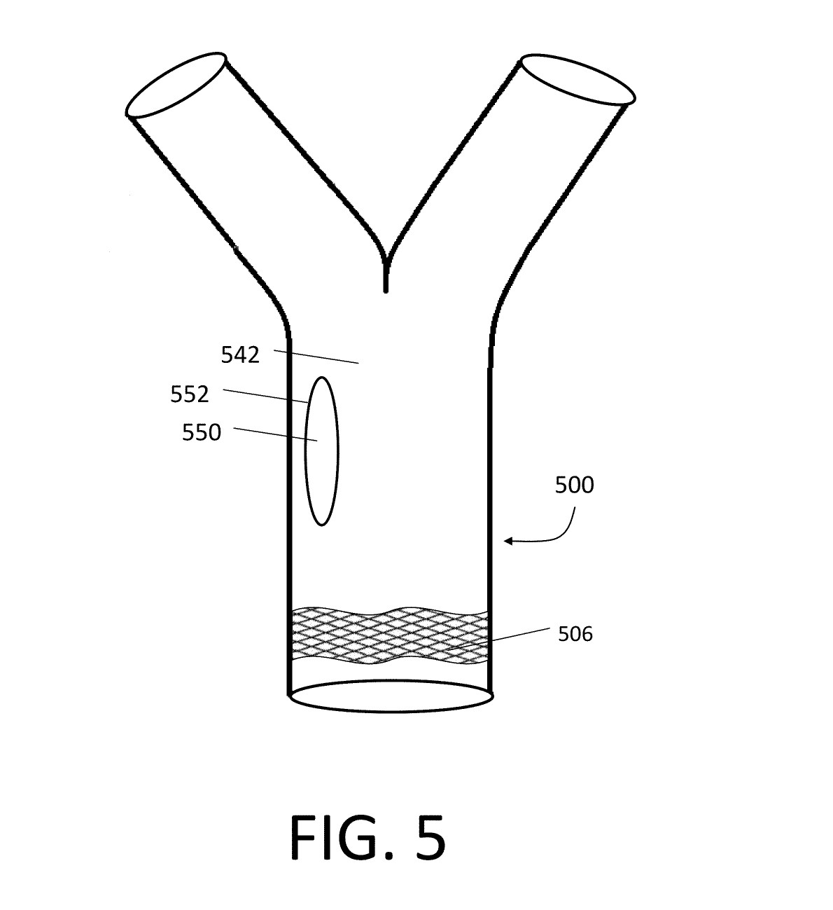

FIG. 5 depicts an embodiment of a branching covered stent-graft 500 with an aperture and a fluid barrier material bonded, affixed, or otherwise coupled to an exterior surface of wired portion 506. In embodiments, a fluid barrier material may be secured to a wire portion by mechanical, chemical or thermal bonding.

Branching covered stent-graft 500 includes third wire portion 506 and trunk 542 with an aligned aperture 550 in both the fluid barrier material of trunk 542 and third wire portion 506. FIG. 5 shows a partial cut away view of the fluid barrier material of trunk 542 to show underlying third wire portion 506. An edge 552 defines the aligned aperture 550. In embodiments, aperture 550 in both the fluid material and wire structure 506 is expected to permit unobstructed blood flow from a tributary blood vessel into or out of branching covered stent-graft 500.

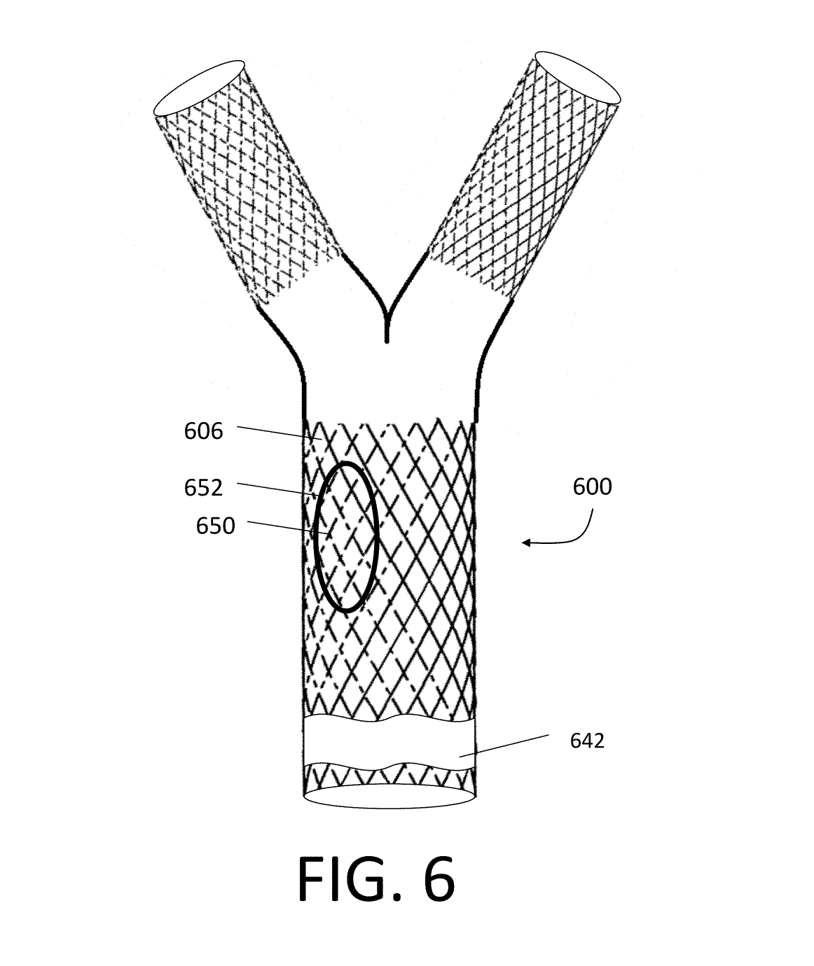

FIG. 6 depicts an embodiment of a branching covered stent-graft 600 with an aperture and a fluid barrier material bonded, affixed, or otherwise coupled to an interior surface of wired portion 606. In embodiments, a fluid barrier material may be secured to a wire portion by mechanical, chemical or thermal bonding.

In embodiments, branching covered stent-graft 600 includes third wire portion 606 and trunk 642 with an aperture 650 in the fluid barrier material of trunk 642. FIG. 6 shows a partial cut away view of the third wire portion 606 to show underlying fluid barrier material of trunk 642. An edge 652 defines aperture 650. Aperture 650 is defined within the fluid barrier material to permit blood flow from a tributary blood vessel into or out of branching covered stent-graft 600, such as to accept blood flow from the azygos vein.

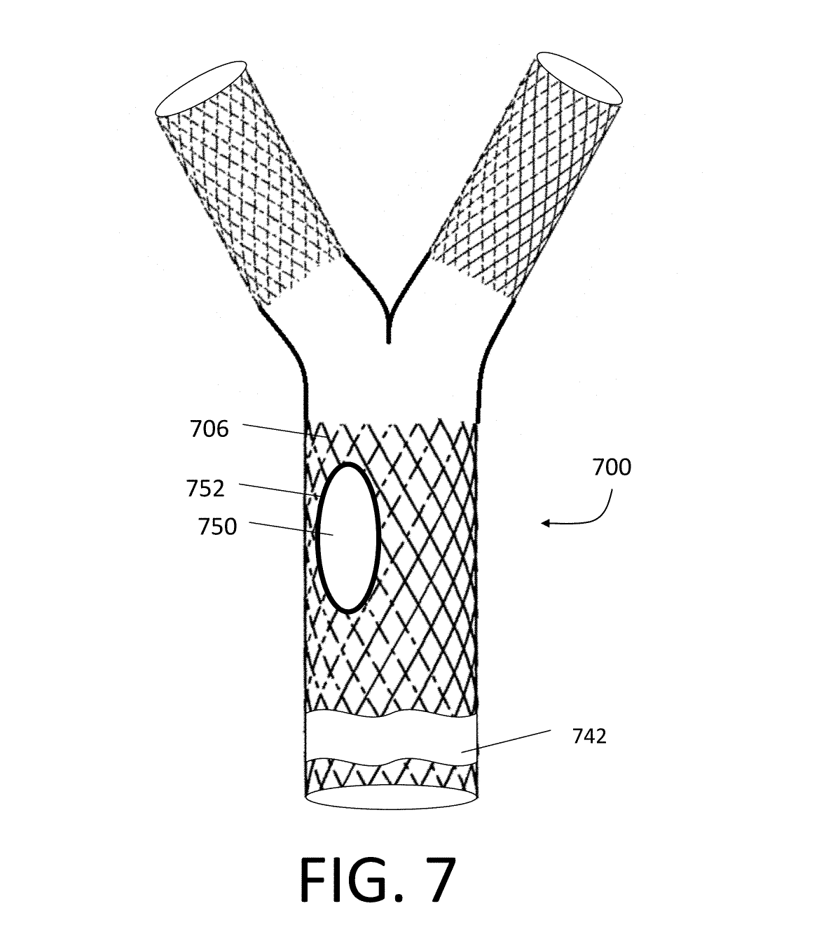

FIG. 7 depicts an embodiment of a branching covered stent-graft 700 with an aperture and a fluid barrier material bonded, affixed, or otherwise coupled to an interior surface of wired portion 706. In embodiments, branching covered stent-graft 700 includes third wire portion 706 and trunk 742 with an aligned aperture 750 in both the fluid barrier material of trunk 742 and third wire portion 706. FIG. 7 shows a partial cut away view of the third wire portion 706 to show underlying fluid barrier material of trunk 742. An edge 752 defines aligned aperture 750. Aperture 750 is defined within the fluid barrier material to permit blood flow from a tributary blood vessel into or out of branching covered stent-graft 700, such as to accept blood flow from the azygos vein.

In embodiments, the distance between an edge of an aperture and a junction of a flexible joint is in the range of 1.0 mm (0.039 inch) to 70 mm (2.76 inches). In embodiments, a long axis of an aperture is in a range of 10 mm (0.394 inches) to 20 mm (0.787 inches) and a short axis of an aperture is in a range of 5 mm (0.197 inch) to 10 mm (0.394 inches). In embodiments, an aperture is located posterior to the plane formed by the first wire portion and the second wire portion, approximating the normal anatomic branching pattern of the SVC. In embodiments, an aperture is in fluid communication with branching covered stent-graft flowpaths. An aperture can allow for preservation of blood flow from important branched or tributary blood vessels as compared to an existing stent-graft without an aperture.

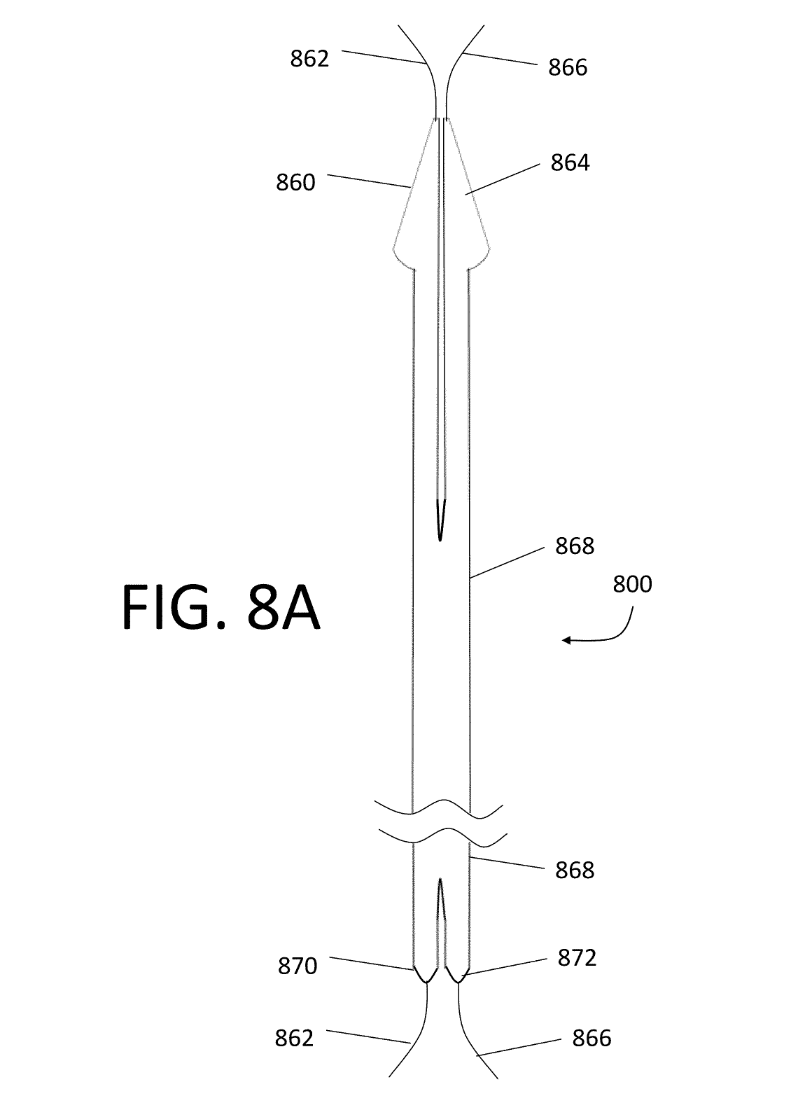

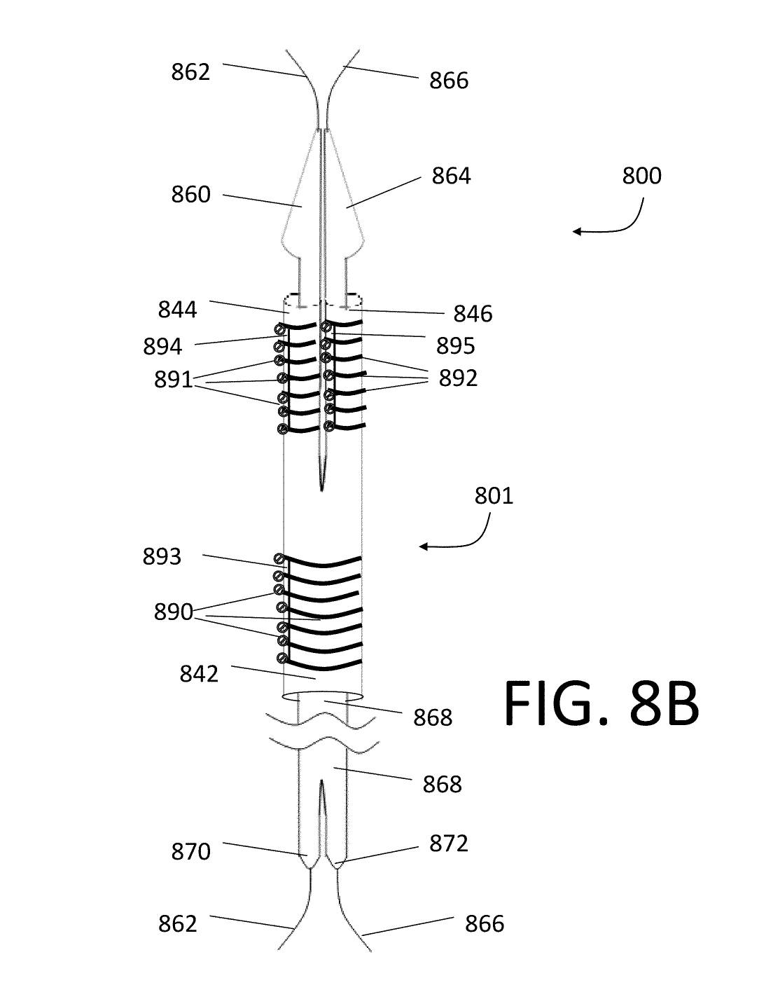

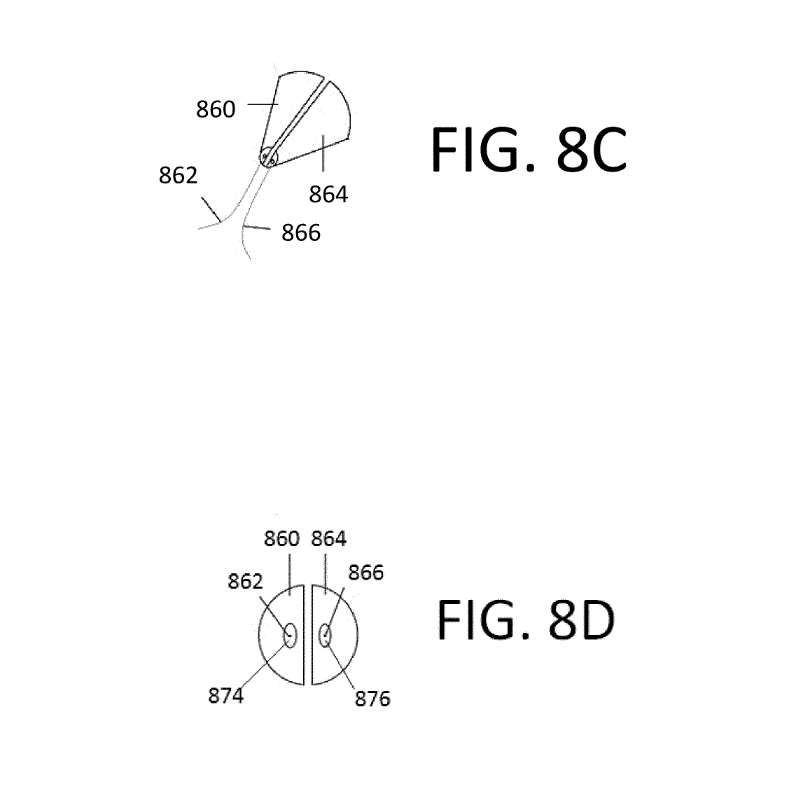

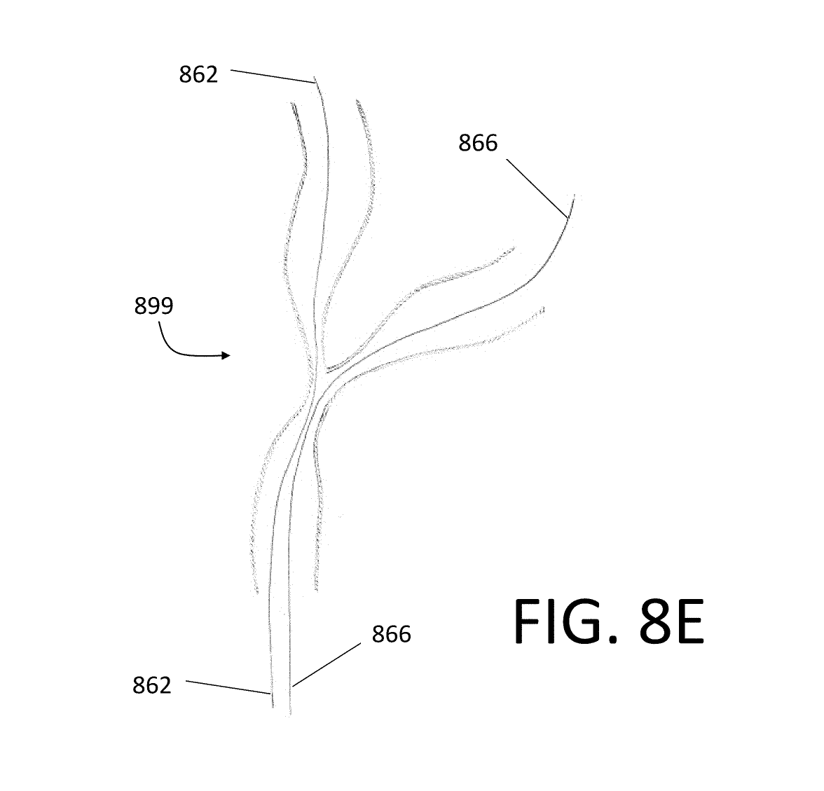

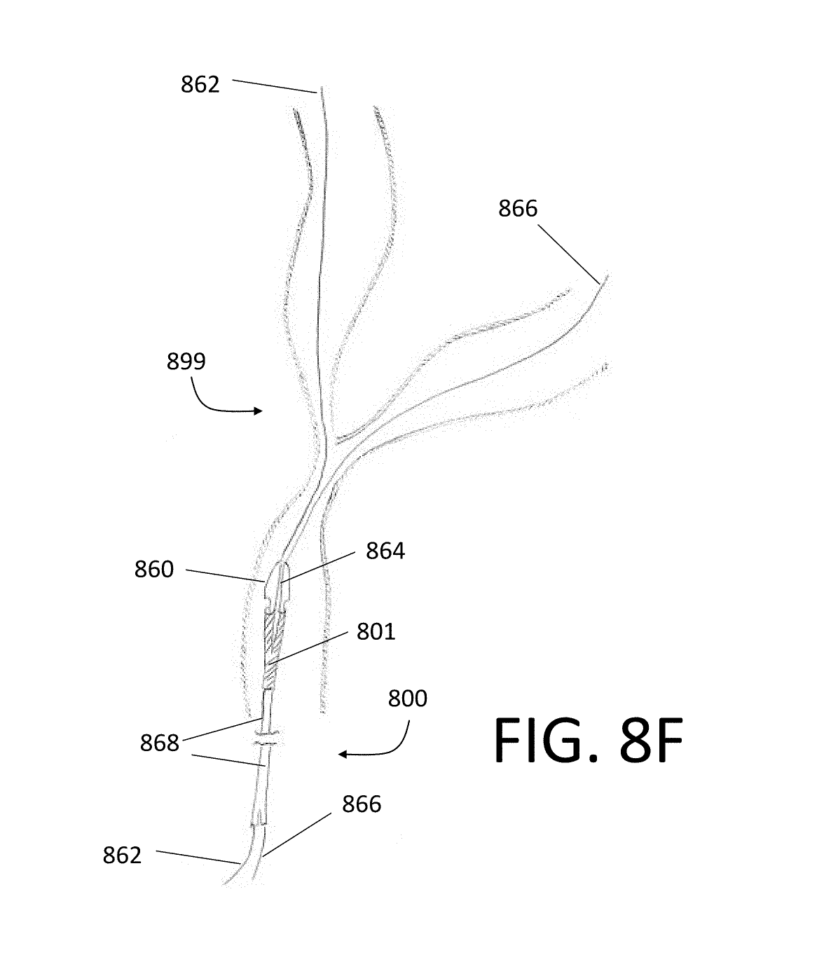

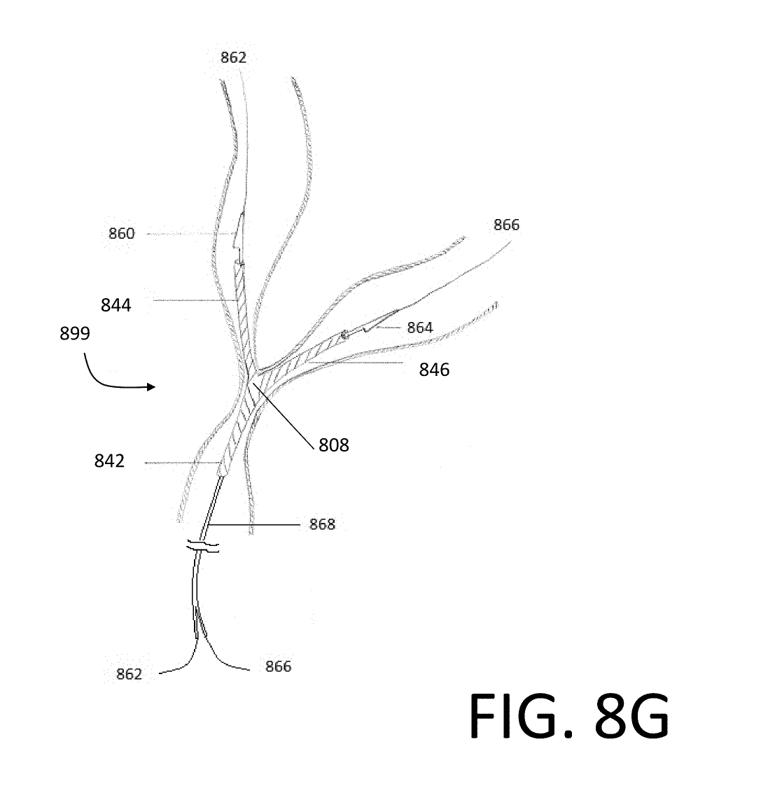

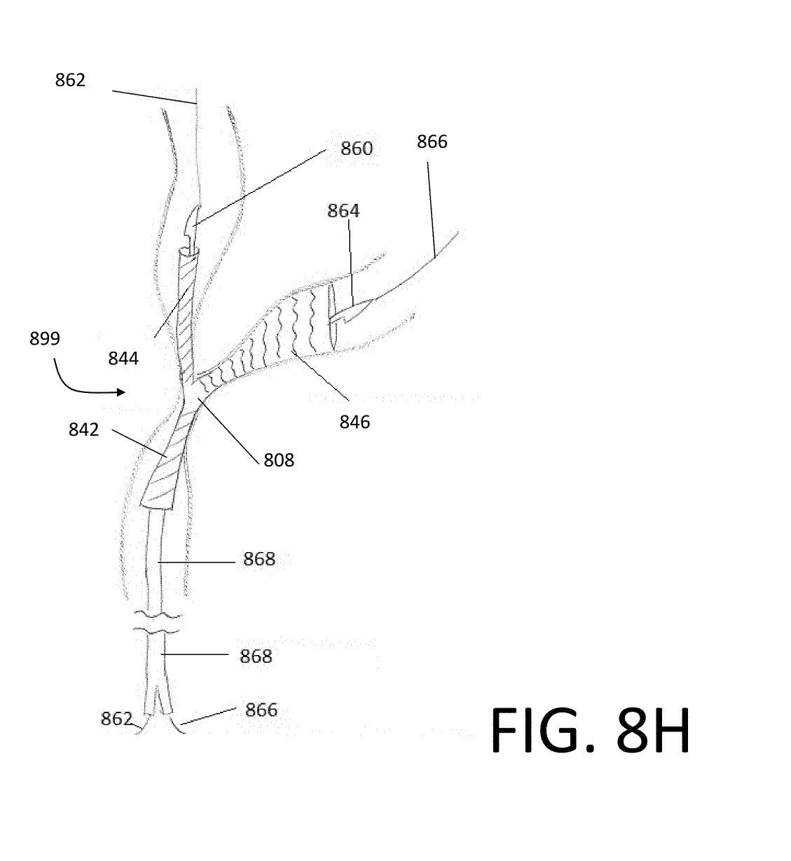

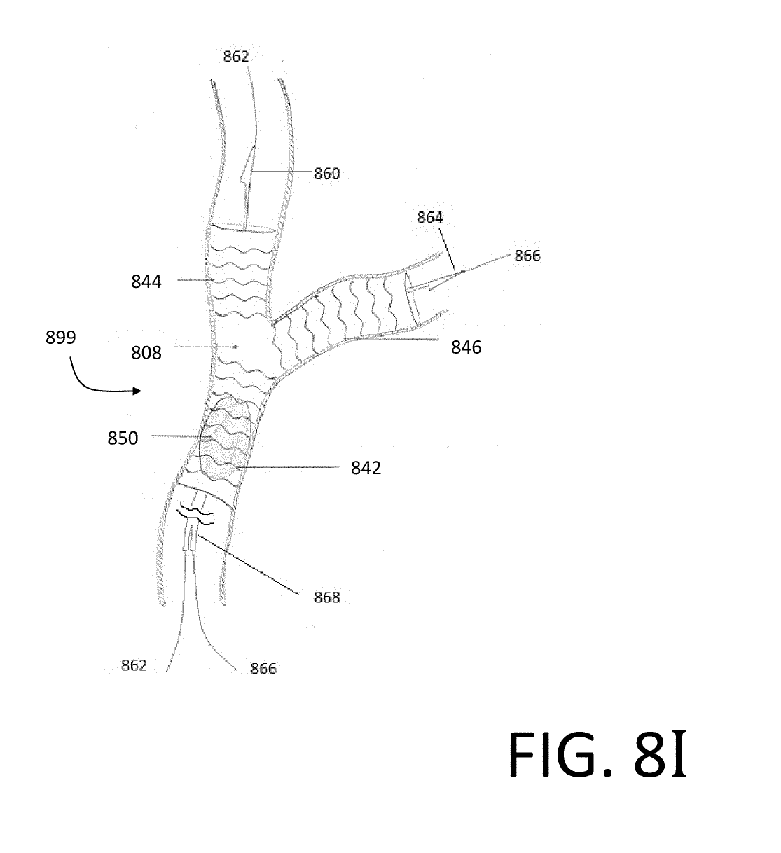

As depicted in FIGS. 8A-8I, a branching covered stent-graft 801 can be deployed by stent-graft delivery system 800. Branching covered stent-graft 801 includes flexible joint 808, trunk 842, first branch 844, second branch 846, and aperture 850.

Referring to FIGS. 8A-8D, Stent-graft delivery system 800 includes first carrier component 860, first guide wire 862, second carrier component 864, second guide wire 866, catheter 868, first back end 870, and second back end 872. In embodiments, carrier components 860, 864 form a split graduated tapered nosecone that provides easier and atraumatic navigation through the vasculature before the vessel bifurcation. In embodiments, carrier components 860, 864 can be made of a polymer that is suitably soft to provide an atraumatic leading edge for delivering branching stent-graft 801. In other embodiments, a carrier component can be formed of a hard plastic such as polycarbonate or metal. In embodiments, catheter 868 is formed by a continuation of the first carrier 860 and second carrier component 864. First carrier component 860 has a lumen 874 movably adjacent to first guide wire 862. Second carrier component 864 has a lumen 876 movably adjacent to second guide wire 866. Catheter 868 defines a central shaft with two lumens, each is contiguous with lumens 874, 876 of carrier components 860 and 864. In embodiments, first guide wire 862 can enter through lumen 874 of first carrier component 860 through a central shaft of catheter 868 and out of first back end 870. In embodiments, second guide wire 866 can enter through lumen 876 of second carrier component 864 through a central shaft of catheter 868 and out of second back end 872. In embodiments, the length of the carrier components 860 and 864 is designed proportional to the length of the first branch 844 and second branch 846. In embodiments, the overall shaft of catheter 868 has length in the range of about 450 mm (17 inches) to about 1000 mm (39 inches).

Referring to FIG. 8B, stent-graft delivery system 800 includes a plurality of individual longitudinally spaced suture loops 890, 891, 892 and pull members 893, 894, 895. Pull member 893 secures suture loops 890 about trunk 842. Pull member 894 secures suture loops 891 about first branch 844. Pull member 895 secures suture loops 892 about second branch 846. The plurality of suture loops 890, 891, 892 is configured to compress branches 844, 846 and trunk 842 in a direction perpendicular to the longitudinal axis of catheter 868. Branches 844, 846 are configured to mount onto carrier components 860, 864 when compressed. Pull members 893, 894, 895 are operably coupled to a control mechanism outside the body (not shown) and are configured to be retracted. Pull members 893, 894, 895 are configured to separate suture loops 890, 891, 892 from about branching covered stent-graft 801 when retracted. Pull members 893, 894, 895 can be retracted individually and in any order. In embodiments, pull members 893, 894, 895 can be manufactured from suture or metal wire or other synthetic material. Branching covered stent-graft 801 is configured to expand from a compressed position to an expanded position when suture loops 890, 891, 892 are separated. The wire portions, which were compressed, will expand to support the wall of the SVC or other blood vessel in which they have been deployed. In embodiments, other delivery systems like delivery system 800 can similarly deploy branching covered stent-graft 300, 400, 500, 600, 700, or other embodiments.

Referring to FIG. 8E, first guide wire 862 and second guide wire 866 are in place in bifurcated or branched blood vessel 899. Referring to FIG. 8F, stent-graft delivery system 800 delivers branching covered stent-graft 801 to bifurcated or branched blood vessel 899 over guide wires 862, 866. In embodiments, first guide wire 862, second guide wire 866, and catheter 868 with carrier components 860, 864 are configured to deliver branching stent-graft 801 to the bifurcation of the SVC. First carrier component 860, with first branch 844 mounted, will follow the path of first guide wire 862. Second carrier component 864, with second branch 846 mounted, will follow the path of second guide wire 866. Branching stent-graft 801 is compressed into a compressed position during delivery.

Referring to FIG. 8G, as stent-graft 801 is advanced over guide wires 862, 866 towards the bifurcation of blood vessel 899, the carrier components 860, 864 and branches 844, 846 will split open as each will follow the path of guidewires 862, 866, respectively. In embodiments, first branch 844 and second branch 846 can be positioned at or near the bifurcated or branched blood vessels. In embodiments, first branch 844 and second branch 846 can be positioned at or near the left or right brachiocephalic veins. In embodiments, trunk 842 can be positioned at or near the SVC. In embodiments, flexible joint 808 can be positioned at or near the bifurcation of the SVC.

Referring to FIGS. 8B and 8H, stent-graft delivery system 800 is configured to allow branching stent-graft 801 to expand from a compressed position to an expanded position when branching stent-graft 801 is positioned at or near a bifurcated or branched blood vessel. Branching stent-graft 801 is configured to expand from a compressed position to an expanded position by removing pull members 893, 894, 895. Removal of pull member 893 separates suture loops 890 from about trunk 842. Trunk 842 is configured to expand upon removal of suture loops 890. Removal of pull member 894 separates suture loops 891 from about first branch 844. First branch 844 is configured to expand upon removal of suture loops 891. Removal of pull member 895 separates suture loops 892 from about second branch 846. Second branch 846 is configured to expand upon removal of suture loops 892. Pull members 893, 894, 895 can be removed independently (i.e., individually and in any order). Referring to FIGS. 8B and 8H, removal of pull member 895 results in the expansion of second branch 846.

Referring to FIGS. 8B and 8I, removal of pull members 893, 894, 895 results in the expansion of branched covered stent-graft 801 from a compressed position to an expanded position. In embodiments, expansion of branching stent-graft 801 into a fully expanded position may be facilitated using a balloon catheter (not shown) subsequent to the deployment of branching stent-graft 801 at the treatment site. In embodiments, deployed branching stent-graft 801 in an expanded position can simultaneously support both vascular walls of the brachiocephalic veins and the vascular walls of the SVC. In embodiments, aperture 850 can be placed at or near a tributary blood vessel. In embodiments, aperture 850 can be placed at or near the azygos vein. Once branched covered stent-graft 801 is deployed in an expanded position, catheter 868 can be retracted and removed from the body. Guide wires 862, 866 can be subsequently removed from the body.

In embodiments, it is expected that branching stent-graft 801 with flexible joint 808 comprised of a fluid barrier material will have increased flexibility as compared to a conventional stent or stent-graft with a joint comprised of a wire portion. It is expected that a fluid barrier will reduce the risk of bleeding from vessel wall injury during catheterization, recanalization, or balloon angioplasty of a chronically narrowed or occluded vessel. It is expected, that a fluid barrier with reduce the risk of restenosis or re-occlusion by inhibiting tissue ingrowth through stent-graft interstices as compared to a stent-graft without such barrier. It is expected that the increased flexibility of flexible joint 808 will allow each of first branch 844, second branch 846, and trunk 842 to be independently adjustable and flexible to fit varied geometries of the blood vessels of patients. In embodiments, an aperture 850 of a branching stent-graft can be positioned at or near the azygos vein. The azygos vein, which transports blood to the SVC, drains itself into the SVC at or near the bifurcation of the SVC. In embodiments, it is expected that an aperture placed at or near the azygos vein will self-align with the azygos vein to allow blood flow from the azygos vein to enter through an aperture 850 into a branching covered stent-graft 801. The flow of blood from the azygos vein into the aperture will cause the self-alignment of the vein with an aperture. It is expected that a branching stent-graft with an aperture placed at or near the azygos vein for the treatment of a stenotic or occlusive lesion will preserve blood flow from the azygos vein into the SVC and reduce the risk of thrombosis as compared to a stent-graft without an aperture placed at or near the azygos vein.

In embodiments, a stent-graft and a stent-graft delivery system can be operably packaged together in a kit. Packaging a stent-graft and stent-graft delivery system together in a kit can facilitate the use of the device by a medical professional for treatment of a patient. A kit can further include instructions for use, such as instructions for operating a stent-graft delivery system to deliver a stent-graft. Such instructions can facilitate the use of a stent-graft and stent-graft delivery systems to deploy a stent-graft in an anatomical structure of the body.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

1. A stent-graft comprising:
a first wire portion defining a first flowpath;
a second wire portion defining a second flowpath;
a third wire portion defining a third flowpath; and
a flexible joint comprising a continuous sheet of fluid barrier material coupled to and interposed between the first wire portion, the second wire portion, and the third wire portion, the flexible joint defining a plenum that is fluidically coupled to each of the first flowpath, the second flowpath, and the third flowpath.

2. The stent-graft of claim 1, wherein the fluid barrier comprises PTFE or PET.

3. The stent-graft of claim 1, wherein the first wire portion, the second wire portion and the third wire portion define an exterior surface coupled to a fluid barrier, the fluid barrier defining a first aperture having an edge that is arranged between about 0.1 cm and about 7 cm from the flexible joint.

4. The stent-graft of claim 3, wherein the third wire portion defines a second aperture that is arranged adjacent to the first aperture of the fluid barrier.

5. The stent-graft of claim 1, wherein the first wire portion, the second wire portion and the third wire portion define an interior surface coupled to a fluid barrier, the fluid barrier defining a first aperture having an edge that is arranged between about 0.1 cm and about 7 cm from the flexible joint.

6. The stent-graft of claim 5, wherein the third wire portion defines a second aperture that is arranged adjacent to the first aperture of the fluid barrier.

7. The stent-graft of claim 6, wherein the second aperture is arranged adjacent to the first aperture between about 0.1 cm and 7 cm from the flexible joint, and wherein the first aperture and the second aperture are configured to align with an azygos vein at or near a superior vena cava.

8. The stent-graft of claim 6, wherein the first wire portion, the second wire portion and the third wire portion define an exterior surface coupled to a fluid barrier, the fluid barrier defining a first aperture having an edge that is arranged between about 0.1 cm and about 7 cm from the flexible joint.

9. The stent-graft of claim 8, wherein the third wire portion defines a second aperture that is arranged adjacent to the first aperture of the fluid barrier.

10. A stent-graft delivery system, comprising:
a control mechanism;
a stent-graft including:
a first wire portion defining a first flowpath, wherein the first wire portion is compressible in a direction perpendicular to the first flowpath;
a second wire portion defining a second flowpath, wherein the second wire portion is compressible in a direction perpendicular to the second flowpath;
a third wire portion defining a third flowpath, wherein the third wire portion is compressible in a direction perpendicular to the third flowpath; and
a flexible joint made of a fluid barrier material, the fluid barrier material being coupled to and interposed between the first, second, and third wire portions;
a catheter having a central shaft, a split graduated nose cone to form a first carrier with a lumen and a second carrier with a lumen, the first carrier and second carrier operably coupled to the stent-graft;
a first guide wire and a second guide wire, the first guide wire movably positioned in the lumen of the first carrier, the second guide wire movably positioned in the lumen of the second carrier;
a plurality of suture loops arranged along the stent-graft and arranged to hold the plurality of suture loops; and
one or more pull members securing the plurality of suture loops about the stent-graft, each pull member operably coupled to the control mechanism, each of the one or more pull members configured to separate suture loops from about the stent-graft when one or more of the longitudinal pull members are released, the stent-graft configured to expand from a compressed state to an expanded state when suture loops are released.

11. The stent-graft delivery system of claim 10, wherein the fluid barrier comprises PTFE or PET.

12. The stent-graft delivery system of claim 10, wherein a first pull member secures suture loops arranged about the first wire portion, a second pull member secures suture loops arranged about the second wire portion, and a third pull member secures suture loops about the third wire portion, and wherein the first pull member, the second pull member, and the third pull member are independently releasable.

14. A kit comprising:
at least one stent-graft comprising:
a first wire portion defining a first flowpath, wherein the first wire portion is compressible in a direction perpendicular to the first flowpath;
a second wire portion defining a second flowpath, wherein the second wire portion is compressible in a direction perpendicular to the second flowpath;
a third wire portion defining a third flowpath, wherein the third wire portion is compressible in a direction perpendicular to the third flowpath; and
a flexible joint made of a fluid barrier material, the fluid barrier material being coupled to and interposed between the first, second, and third wire portions;
a stent-graft delivery system comprising:
a control mechanism;
a catheter having a central shaft, a split graduated nose cone that forms a first carrier with a lumen and a second carrier with a lumen, the first carrier and second carrier operably coupled to the stent-graft;
a first guide wire and a second guide wire, the first guide wire movably positioned in the lumen of the first carrier, the second guide wire movably positioned in the lumen of the second carrier;
a plurality of suture loops arranged along the stent-graft and arranged to hold the plurality of suture loops; and
one or more pull members securing the plurality of suture loops about the stent-graft, each pull member operably coupled to the control mechanism, each of the one or more pull members configured to separate suture loops from about the stent-graft when one or more of the longitudinal pull members are released, the stent-graft configured to expand from a compressed state to an expanded state when suture loops are released.

15. The kit of claim 14, wherein the fluid barrier comprises PTFE or PET.

16. The kit of claim 14, wherein the first wire portion, the second wire portion and the third wire portion define an exterior surface coupled to a fluid barrier, the fluid barrier defining a first aperture having an edge that is arranged between about 0.1 cm and about 7 cm from the flexible joint.

17. The kit of claim 16, wherein the third wire portion defines a second aperture that is arranged adjacent to the first aperture of the fluid barrier.

18. The kit of claim 17, wherein the first wire portion, the second wire portion and the third wire portion define an interior surface coupled to the fluid barrier.

19. The kit of claim 14, wherein the third wire portion defines a second aperture that is arranged adjacent to a first aperture of a fluid barrier between about 0.1 cm and 7 cm from the flexible joint, and wherein the first aperture and the second aperture are configured to align with an azygos vein at or near a superior vena cava, the kit further comprising a first pull member that secures suture loops arranged about the first wire portion, a second pull member that secures suture loops arranged about the second wire portion, and a third pull member that secures suture loops about the third wire portion, and wherein the first pull member, the second pull member, and the third pull member are independently releasable.
